(19) United States
(12) Patent Application Publication
(10) Pub. No.: US 2004/0044338 A1
(43) Pub. Date: Mar. 4, 2004

(54) Methods and systems for administering microcurrent therapy for treatment and prevention of side effects associated with cancer treatment

(76) Inventors: Arlene J. Lennox, Elburn, IL (US); Sandra Funder, Crown Point, IN (US)

Correspondence Address: Luis M. Ortiz, Ortiz & Lopez, PLLC, P.O. Box 4484, Albuquerque, NM, 87196, US

(21) Appl. No.: 10/651,501

(22) Filed: Aug. 29, 2003

Related U.S. Application Data
(60) Provisional application No. 60/407,093, filed on Aug. 30, 2002.

(51) Int. Cl. A61B 18/04

(52) U.S. Cl. 606/32

(57) ABSTRACT

Methods and systems apply biphasic DC signal at various frequencies, durations and/or amplitudes to areas of a cancer patient's body being targeted for preventing or alleviating cancer-therapy side effects. Microcurrent therapy can be provided prior to, during and after procedures used to treat cancer. During microcurrent treatment a biphasic DC signal can be applied from a microcurrent therapy system to an area of the cancer patient's body targeted for microcurrent treatment using at least one electrode. The electrode can be actively manipulated on the treatment area for a predetermined time. The system can provide impedance matching between it and the patient during treatment. A feedback module provides a controller impedance information between the patient and system where a mismatch is used to adjust the system to overcome impedance variations that occur between said system and said patient's body during the predetermined time of treatment.

### PRIORITY TO PREVIOUSLY FILED APPLICATION

This application claims the benefit of priority to a provisional patent application, Ser. No. 60/407,093, filed on Aug. 30, 2002, and entitled “methods and systems for administering and monitoring results of microcurrent therapy administered to cancer patients for treatment and prevention of side effects associated with cancer treatment.”

### STATEMENT OF GOVERNMENT INTEREST

The United States government has certain rights to the present disclosure in accordance with contract DE-AC02-76CH03000 with the U.S. Department of Energy.

### FIELD OF THE INVENTION

The present invention generally relates to treatments that address the side effects of cancer therapy. The present also relates to the administration of microcurrent therapeutic techniques for relieving the toxicities associated with cancer therapy. More particularly, the present invention is related to methods and systems for administering, and monitoring, microcurrent therapy provided to cancer patients for the treatment and prevention of at least one of many medical conditions associated with cancer and its treatment. Medical conditions subject to treatment under the present invention can include those associated with radiation treatment, chemotherapy treatment and cancer-related surgery, including: radiation-induced fibrosis; xerostomia; trismus; proctitis (including associated diarrhea); nausea; limited range of motion; loss of motor coordination; edema, lymphedema, scar tissue, and trismus.

### BACKGROUND OF THE INVENTION

All forms of cancer treatment are associated with some type of side effect. Scars caused by surgery, and nausea and/or hair loss caused by chemotherapy tend to be self limiting once the cancer treatment is completed. However, while the acute effects of radiation therapy tend to improve with time, the late effects of radiation, especially fibrosis, can continue to worsen with time.

Radiation therapy uses penetrating beams of radiation to treat or remediate disease. Various forms of radiation therapy, including photon, electron, and neutron radiation, are used on a daily basis in the United States and throughout the world. One major use of radiation therapy is in the treatment of cancerous tumors. The basic effect of radiation therapy is to destroy the ability of cells to divide and grow by damaging their DNA strands. This effect is useful in killing cancerous cells, but also has the disadvantage of damaging healthy tissue. As a result, a patient may be required to live with debilitating side effects of cancer treatment including limb or organ swelling, thickening and hardening of otherwise normal tissue, and chronic or constant pain.

The deleterious side effects produced in the patient as a result of radiation therapy are known as radiation toxicities. Radiation toxicities are associated with any ionizing radiation treatment and include fibrotic tissue (scar tissue), xerostomia (loss of salivary function), trismus (closure of the jaw), radiation proctitis (inflammation of the rectum), limited range of motion, loss of motor coordination, edema (swelling), and lymphedema (swelling resulting from obstruction of the lymphatic vessels or lymph nodes). Unfortunately, late effects associated with radiation therapy are progressive and in most cases will tend to worsen over time. Current practices for treating late side effects of cancer treatment include physical therapy, massage, exercise, and drugs such as diuretics, painkillers, steroids, and saliva inducers. In most cases, however, such treatments can only provide patients with minimal relief. Patients will most likely be required to live with the debilitating side effects of radiation therapy or the chemical and/or biological side effects of medicinal therapies used to treat radiation side effects.

For example, salivary glandular tissue is often included in radiation treatment fields involving head and neck cancer. Damage to saliva glands can cause xerostomia (dry mouth), leading to an increase in dental caries, oral yeast infections, and difficulty in digesting food. Patients suffering from xerostomia frequently carry water bottles or use sour candy to keep their mouths moist. The most common medication for relieving dry mouth is pilocarpine hydrochloride. Patients using pilocarpine must take it daily for about 90 days to achieve improvement and they lose the benefit when they stop taking the drug. The cost of the drug as well as the side effects of sweating and gastrointestinal distress, often cause patients to discontinue use of the drug.

The use of electrical stimulation for simple pain relief, not associated with cancer or radiation therapy, has been well established by physical therapy centers. Physical therapists use microcurrent therapy in a variety of ways for the treatment of pain not associated with cancer or radiation treatment, often in combination with massage, heat and physical manipulation. There are many commercially electrical stimulation devices marketed for the treatment of pain relief, most of which are commonly referred to as TENS (transcutaneous electrical nerve stimulation) units. Typical TENS units emit electrical pulses with alternating positive and negative polarities in the 10 to 500 kilohertz (KHz) range and currents in the milliampere (mA) range. Microcurrent (μA) units are often incorrectly referred to as TENS units, but microcurrent units deliver lower currents (microampere range) and lower frequencies (0.5 to several hundred Hertz). In general, units using higher current and frequencies are more effective at blocking acute pain, but the pain relief is not lasting. By contrast, microcurrent therapy using lower frequencies requires longer treatment times to achieve pain relief, but the relief can endure for many hours after the treatment has terminated.

The present inventor has previously recognized that a need existed in the medical profession for a more effective means of alleviating radiation toxicities typically associated with radiation therapy. In U.S. Pat. No. 6,115,637, entitled “microcurrent therapeutic technique for treatment of radiation toxicity,” issued Sep. 5, 2000, and herein incorporated by reference, the present inventor previously disclosed methods for alleviating radiation toxicities associated with radiation therapy. As described in the patent, a sinusoidally pulsed biphasic DC signal can be applied to an affected bodily area using at least one electrode. The electrode can be manipulated using active tactile manipulation for a predetermined time and the frequency of the sinusoidally pulsed biphasic DC signal can be adjusted during the course of the treatment. The patent also describes a method of applying a spiked pulsed biphasic DC signal to an affected bodily area using at least one electrode. The electrode can also be manipulated using active tactile manipulation for a predetermined time and the frequency of the spiked pulsed biphasic DC signal can also be adjusted during the course of the treatment.

The microcurrent therapeutic techniques described in the '637 patent are used to alleviate debilitating radiation toxicities associated with radiation therapy, including fibrotic tissue, xerostomia, trismus, radiation proctitis, limited range of motion, loss of motor coordination, edema, and lymphedema. An objective set forth in the '637 patent is to provide greater pain relief than prior known methods for treating the late side effects of cancer treatment, thus allowing patients to have higher qualities of life. The patent also describes use of methods for pre-treating a patient in an effort to avoid the radiation toxicities associated with radiation therapy.

Since disclosing the patented use of microcurrent therapy treatment of toxicities associated with radiation cancer treatment, the present inventors have determined that a need still exists for disclosure of new and improved treatment regimes, via improved methods and systems, that can be used to accurately target and treat, pre-treat, and concurrently treat areas on the human body that can fall ill as a result of cancer therapies. The present inventors therefore now disclose new methods and systems for managing medical conditions associated with cancer treatment including: medical conditions associated with radiation treatment, chemotherapy, and cancer-specific surgery. Medical conditions subject to treatment include: radiation-induced fibrosis; xerostomia; trismus; surgical scars; proctitis (diarrhea); nausea associated with radiation treatment and chemotherapy; limited range of motion; loss of motor coordination; edema, and lymphedema. The new methods described herein include methods for applying microcurrent treatment before cancer therapy, concurrent with cancer therapy and after cancer therapy in order to minimize side effects.

### SUMMARY OF THE INVENTION

The following summary of the invention is provided to facilitate an understanding of some of the innovative features unique to the present invention and is not intended to be a full description. A full appreciation of the various aspects of the invention can only be gained after considering the entire specification, claims, drawings and abstract as a whole.

Side effects suffered by cancer treatment patients can be prevented and/or treated by the application of microcurrent therapeutic techniques. Set forth herein are methods and systems for applying biphasic DC signal at various frequencies and amplitudes to areas of a cancer patient's body being targeted for microcurrent therapy. The methods and systems described herein are effective for relieving toxicities associated with cancer therapy. Spiked and/or sinusoidally pulsed biphasic DC signal can be applied to the affected bodily area using active tactile manipulation by at least one electrode for a predetermined time and adjusting the frequency range during the course of the pre-treatment, concurrent treatment and/or post-treatment of radiation therapy cancer patients.

A system used to provide DC signal and frequency signaling can include impedance matching capabilities, wherein system output is adjusted to changes in human body impedance during treatment. The biphasic DC signal can be applied to the patient's body using at least one electrode for a predetermined time. Obviously use of at least one electrode implies that the patient is electrically grounded by means known in the art (e.g., conductive cuffs, conductive-adhesive pads, conductive plates, liquid baths, etc.).

The frequency of the biphasic DC signal used during treatment can be adjusted within a range of from about 0.5 Hz to about 500 Hz. The biphasic DC signal treatment can be applied for a predetermined time, preferably for about 20 minutes per treatment mode (e.g., spiked and sinusoidal). Current used during treatment can range from about 25-600 microamps.

Preferably, the size of the electrodes used to apply both the sinusoidally pulsed biphasic DC signal and the spiked pulsed biphasic DC signal will be selected so as to achieve maximum skin contact over the largest possible area of the body being targeted for treatment. Electrodes used to apply the sinusoidally and spiked pulsed biphasic DC signal can be provided in the form of any of a: probe, roller, adhesive pad, plate, cuff, clamp, sheet, and other conductive media known in the art. The electrodes used to apply both currents can also be selected for treating larger affected bodily areas, smaller affected bodily areas, or crevicular areas. Electrodes can also be cylindrical in shape or can be designed to be suitable for intra-oral manipulation and rectal and/or vaginal insertion.

### BRIEF DESCRIPTION OF THE DRAWINGS

The accompanying figures, in which like reference numerals refer to identical or functionally-similar elements throughout separate views and which are incorporated in and form part of the specification, further illustrate the present invention and together with the detailed description of the invention serve to explain the principles of the present invention.

FIG. 1 is an illustration of a flow chart of a method for administering microcurrent therapy to a patient;

FIG. 2 is an illustration of another flow chart of a method for administering microcurrent therapy to a patient;

FIG. 3 is an illustration of yet another flow chart of a method for administering microcurrent therapy to a patient;

FIG. 4 is an illustration of another flow chart of a method for administering microcurrent therapy to a patient;

FIG. 5 is an illustration of a system that can be used for providing microcurrent for patient therapy in accordance with embodiments the present invention;

FIG. 6 is an illustration of an electrotherapy treatment technique where a microcurrent therapy patient's hands are allowed to rest on large metal plates while impedance-controlled microcurrent therapy is delivered using a metal roller;

FIG. 7 is an illustration of a flow chart for administering sinusoidal microcurrent therapy to a patient suffering from neck and/or head fibrosis;

FIG. 8 is an illustration of a flow chart for administering spiked microcurrent therapy to a patient suffering from neck and/or head fibrosis;

FIG. 9 is a graphical illustration of the range of cervical rotation over time for three microcurrent therapy patients initially experiencing severe range-of-motion limitations;

FIG. 10 is a graphical illustration of the range of cervical extension/flexion over time for three microcurrent therapy patients initially experiencing severe range-of-motion limitations;

FIG. 11 is a graphical illustration of range of cervical lateral flexion over time for four microcurrent therapy patients initially experiencing severe range-of-motion limitations;

FIG. 12 is an illustration of a flow chart for administering sinusoidal microcurrent therapy to a patient suffering from xerostomia;

FIG. 13 is an illustration of a flow chart for administering spiked microcurrent therapy to a patient suffering from xerostomia;

FIG. 14 illustrates the use of a Therabite scale to measure the oral opening ability of a patient's mouth.

### DETAILED DESCRIPTION

The particular values and configurations discussed in these nonlimiting examples can be carried and are cited merely to illustrate at least one embodiment of the present invention and are not intended to limit the scope of the invention.

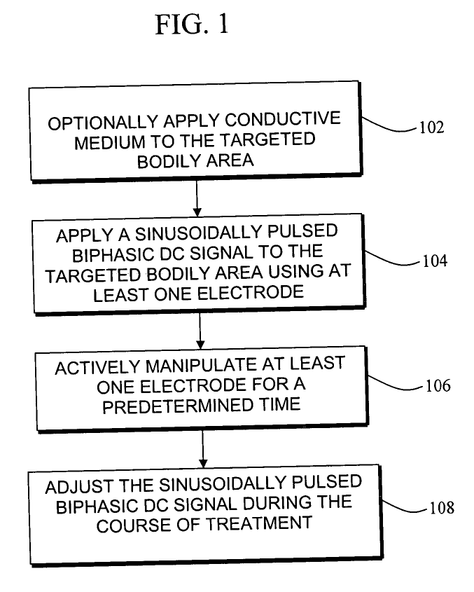

Referring to FIG. 1, a flow diagram is provided that illustrates steps of a treating a patient with microcurrent therapy using a sinusoidally pulsed biphasic DC signal. Prior to treatment, a conductive medium such as a conductive gel or saline solution can optionally be applied to the affected bodily area on the patient, as shown in block 102. Conductive media can enhance the transfer of current and associated signals to a patient. Some applications may not require conductive media such as conductive-adhesive pads, which already include a conductive medium. Conductive gel can enhance the transfer of current and associated signals through the patient. As shown in block 104, a sinusoidally pulsed biphasic DC signal can be applied to the affected bodily area using at least one electrode. The at least one electrode can be actively manipulated for a predetermined time during treatment, as shown in block 106.

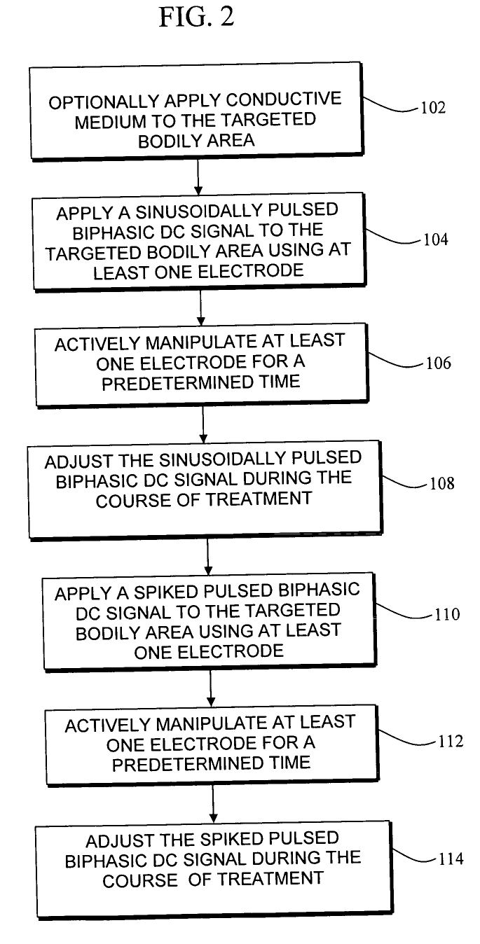

It should be appreciated that a patient can be placed into contact with electrodes that provide passive exposure to microcurrent; however, the best results for microcurrent treatment have been demonstrated through active manipulation of at least one electrode. It should be further appreciated that block 106 can also refer to treatment wherein the time period between manipulation can be on the order of several minutes. Although treatment times can vary, treatment for about 20 minutes has proven effective in prior testing by the inventors. The frequency and amplitude of the sinusoidal pulsed biphasic DC signal can be adjusted during the course of treatment, as shown in block 108. A frequency range shown effective in prior testing is from about 0.5 Hz up to about 500 Hz. Current can range from 25 up to about 600 microamps. Frequency and amplitude adjustments can be made downward or upward. Referring to FIG. 2, a flow diagram is provided that illustrates steps of a treating a patient with microcurrent therapy using both spiked and sinusoidally pulsed biphasic DC signals. Prior to treatment, a conductive gel can be optionally applied to the targeted bodily area on the patient, as shown in block 102. As shown in block 104, a sinusoidally pulsed biphasic DC signal can be applied to the affected bodily area using at least one electrode. The at least one electrode can be actively manipulated for a predetermined time during treatment, as shown in block 106. It should be appreciated that a patient can be placed into contact with electrodes that provide passive exposure to microcurrent; however, the best results for microcurrent treatment have been demonstrated through active manipulation of at least one electrode. It should be further appreciated that block 106 can also refer to treatment wherein the time period between manipulation can be on the order of several minutes. Although treatment times can vary, treatment for about 20 minutes has proven effective in prior testing by the inventors.

The frequency and amplitude of the sinusoidal pulsed biphasic DC signal can be adjusted during the course of treatment, as shown in block 108. A frequency range shown effective in prior testing is from about 0.5 Hz to about 500 Hz. Current can range from 25 up to about 600 microamps. Adjustments can be made downward or upward. Prior to ongoing treatment, a conductive gel can be re-applied to the affected bodily area on the patient after block 108, as previously described with respect to block 102. As shown in block 110, a spiked pulsed biphasic DC signal can be applied to the affected bodily area using at least one electrode. The at least one electrode can be actively manipulated for a predetermined time during treatment, as shown in block 112. Although treatment times can vary, treatment for about 20 minutes has proven effective in prior testing by the inventor. The frequency and amplitude of the spiked pulsed biphasic DC signal can be adjusted during the course of treatment, as shown in block 114. As with the sinusoidal biphasic signal, a frequency range shown effective in prior testing is from about 0.5 Hz up to about 500 Hz. Current can range from 25 up to about 600 microamps. Frequency and amplitude adjustments can be made downward or upward.

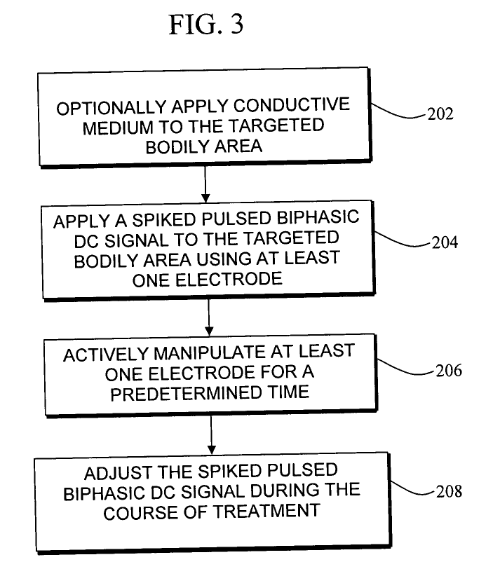

Referring to FIG. 3, a flow diagram is provided that illustrates steps of a treating a patient with microcurrent therapy. Prior to treatment, a conductive gel can optionally be applied to the affected bodily area on the patient, as shown in block 202. As shown in block 204, a spiked pulsed biphasic DC signal can be applied to the affected bodily area using at least one electrode. The at least one electrode can be actively manipulated for a predetermined time during treatment, as shown in block 206. It should again be appreciated that a patient can be placed into contact with electrodes that provide passive exposure to microcurrent; however, the best results for microcurrent treatment have been demonstrated through active manipulation of at least one electrode. It should be further appreciated that block 206 can also refer to treatment wherein the time period between manipulation can be on the order of several minutes. Although treatment times can vary, treatment for about 20 minutes has proven effective in prior testing by the inventor. The frequency and amplitude of the spiked pulsed biphasic DC signal can be adjusted during the course of treatment, as shown in block 208. A frequency range shown effective in prior testing is from about 0.5 Hz to about 500 Hz. Current can range from 25 microamps up to about 600 microamps. Adjustments can be made downward or upward.

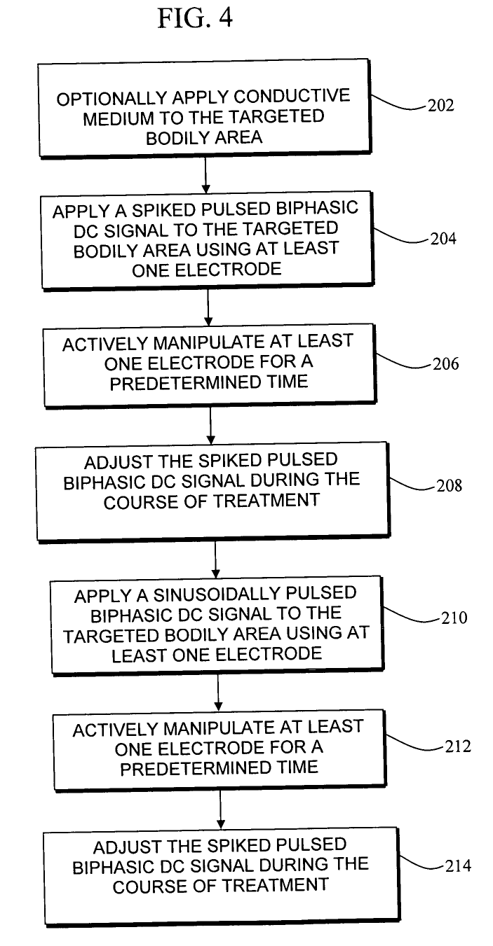

Referring to FIG. 4, a flow diagram is provided that illustrates steps of a treating a patient with microcurrent therapy using both spiked and sinusoidally pulsed biphasic DC signals. Prior to treatment, a conductive gel can be optionally applied to the affected bodily area on the patient, as shown in block 202. As shown in block 204, a spiked pulsed biphasic DC signal can be applied to the affected bodily area using at least one electrode. The at least one electrode can be actively manipulated for a predetermined time during treatment, as shown in block 206. It should yet again be appreciated that a patient can be placed into contact with electrodes that provide passive exposure to microcurrent; however, the best results for microcurrent treatment have been demonstrated through active manipulation of at least one electrode. It should be further appreciated that block 206 can also refer to treatment wherein the time period between manipulation can be on the order of several minutes. Although treatment times can vary, treatment for about 20 minutes has proven effective in prior testing by the inventor. The frequency and amplitude of the spiked pulsed biphasic DC signal can be adjusted during the course of treatment, as shown in block 208. A frequency range shown effective in prior testing is from about 0.5 Hz to about 500 Hz. Current can range from 25 up to about 600 microamps. Adjustments can be made downward or upward. After treatment with the spiked pulsed biphasic DC signal is completed, treatment using a sinusoidally pulsed biphasic DC signal can be provided.

A conductive gel can be applied to the affected bodily area on the patient prior to sinusoidal treatment shown in block 210, as previously described with respect to block 202. Conductive gel can enhance the transfer of current and associated signals through the patient. As shown in block 210, a sinusoidally pulsed biphasic DC signal can be applied to the targeted bodily area using at least one electrode. The at least one electrode can be actively manipulated for a predetermined time during treatment, as shown in block 212. Although treatment times can vary, treatment for about 20 minutes has been proven effective in prior testing by the inventors. The frequency and amplitude of the sinusoidal biphasic DC signal can be adjusted as shown in block 214. As with the spiked biphasic signal, the frequency range of the sinusoidal biphasic signal that has been shown effective in prior testing is from about 0.5 Hz up to about 500 Hz. Current can range from 25 to about 600 microamps. Frequency and amplitude adjustments can be made downward or upward.

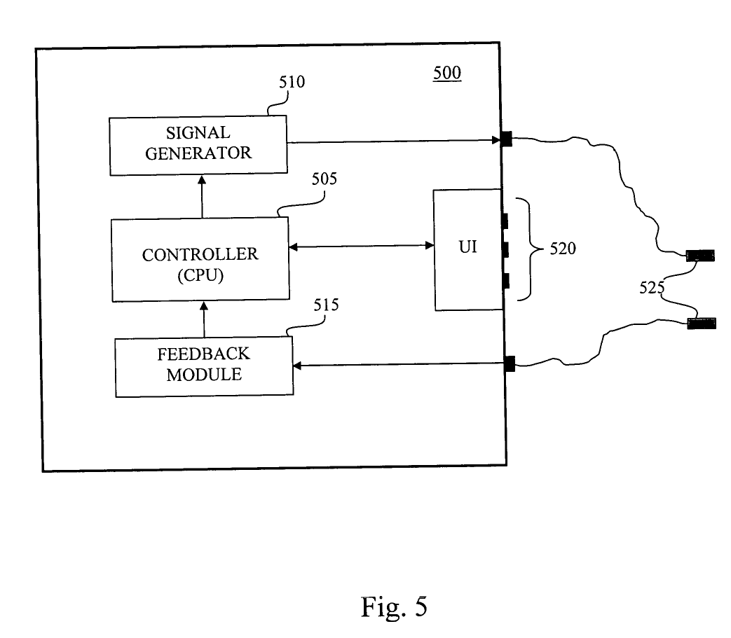

It is well known that the body's impedance changes when electrical current passes through it. The more sophisticated devices used for providing microcurrent therapy for simple pain relief contain circuitry that monitors impedance and adjusts the output current to compensate for changes. Such devices can also deliver fast rise time pulses that can affect voltage-sensitive sodium and calcium ion channels. Referring to FIG. 5, a block diagram of a system 500 that can be used to deliver impedance-controlled microcurrent therapy for cancer/radiation patients is shown. The system 500 can include a controller 505, a signal generator 510 capable of providing a broad range of signal-types, frequencies and amplitudes consistent with the provision of microcurrent therapy as described further herein, and a user interface 520. Signals provided by the signal generator 510 will preferably be biphasic direct current (DC) signals. The system 500 can be adjusted at the user interface (UI) 520 to deliver signals having fast rise time pulses from the signal generator 510. The system 500 can also include a feedback module 515 for measuring impedance variations between it and a human body during microcurrent therapy. Impedance variations acquired by the feedback module can be used by the controller 505 to adjust signals rendered by the signal generator 510. The system 500 can adjust the effectiveness of its signals provided to target bodily areas of a cancer patient using electrodes 525. Cancer/radiation patients can be treated using microcurrent methods described herein via conductive electrodes 525, which can also be provided in the form of probes (among other media).

Patients who experience late effects of radiation therapy for head-and-neck cancer can be treated with microcurrent therapy systems and methods. Objective range-of-motion (ROM) measurements can be carried out for cervical rotation, extension/flexion, and lateral flexion before therapy, during and after therapy using the monitoring system described herein. The present inventor has developed methods of treatment using microcurrent systems and has also developed and used a measuring system to monitor patient improvement at the end of each microcurrent treatment. Treatment can preferably be provided using impedance-controlled systems.

At the end of a course of microcurrent therapy, 92% of twenty-six patients in a study conducted by the present inventor exhibited improved cervical rotation, 85% had improved cervical extension/flexion, and 81% had improved cervical lateral flexion. Of patients returning for a three-month follow-up therapy, 91% maintained cervical rotation range of motion greater than their pre-therapy measurements. Eighty-two percent maintained improved cervical extension/flexion, and 77% maintained improved lateral flexion. When the range-of-motion measurements were stratified by pretreatment severity (severe, moderate, mild, or a-symptomatic) it was observed that the degree of improvement directly correlated with severity. Patients who had more severe initial symptoms experienced a higher percentage of improvement than those with milder symptoms. For these patients the cervical rotation ROM changed from a baseline of 59±12 degrees to 83±14 degrees at three months; flexion/extension improved from 47±10 to 73±13 degrees; and lateral flexion went from 31±7 to 48±9 degrees. Some patients also reported improvement in symptoms such as tongue mobility, facial asymmetry, xerostomia, cervical/facial muscle spasms, trismus, and soft-tissue tenderness. No adverse effects resulting from the microcurrent therapy were observed.

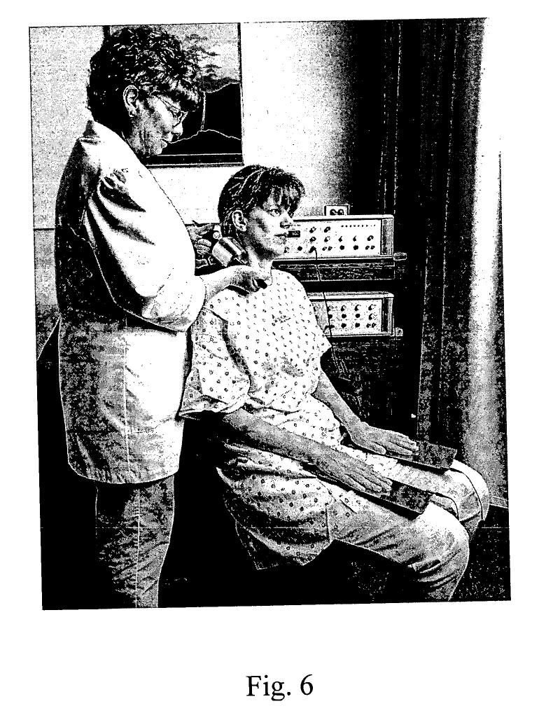

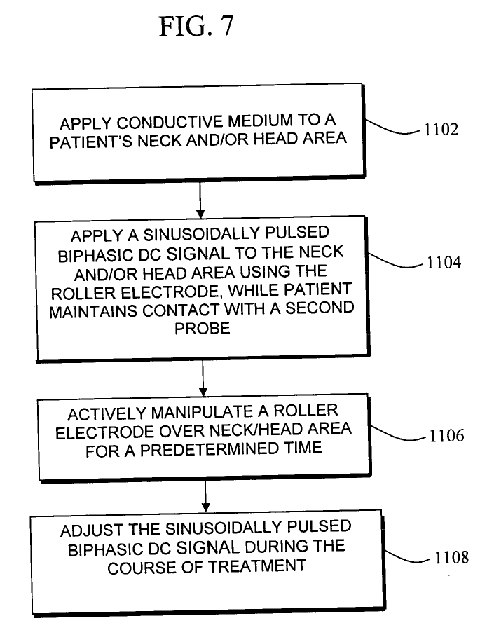

FIG. 6 shows a treatment technique for applying microcurrent therapy to a patient. Referring to FIG. 7, a flow diagram sets forth steps that can be taken to apply microcurrent therapy to patient suffering from fibrosis of the neck and/or head. A system such as that described in FIG. 5 can be used for delivering microcurrent up to about 600 microamps, at various frequencies (e.g., spiked/sinusoidal pulsed biphasic) in the range of 0.5 Hz to 500 Hz, and at various durations (e.g., about 20 minutes).

As shown in block 1102 of FIG. 7, a conductive medium can be applied to the patient's neck and or head area in order to enhance conductivity. Then as shown in block 1104, a sinusoidally pulsed biphasic DC signal can be applied to the neck/head areas using a roller-type electrode, although it should be appreciated that other conductive electrodes can be used. The roller electrode should preferably be smooth enough, or rotate freely enough, to be maneuvered comfortably over a patient's neck area. As shown in block 1106, the electrode (probe) can be manipulated over the patient's neck for a predetermined time. Generally a 10-20 minute treatment is within a comfortable range for most patients. Finally, as shown in block 1108 the frequency of the sinusoidal signal can be adjusted during the course of treatment. Frequency an be varied within a 0.5-500 Hz range, and applied current during treatment can be up to about 600 microamps.

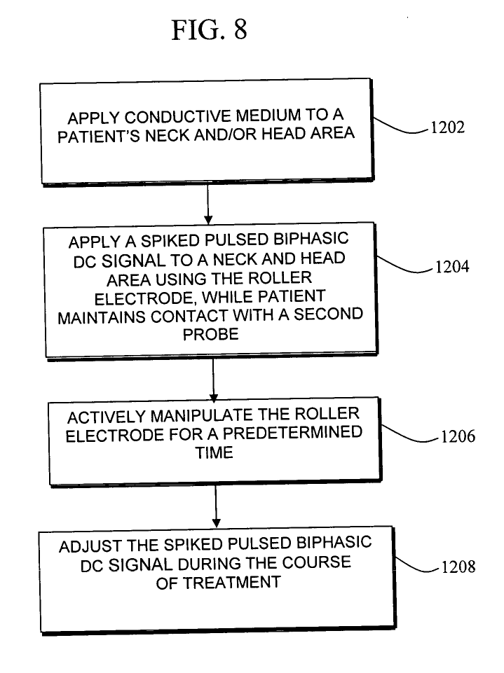

Referring to FIG. 8, a flow diagram sets forth steps that can be taken to apply microcurrent therapy to patient suffering from fibrosis of the neck and/or head. As shown in block 1202, a conductive medium can be applied to the patient's neck and or head area in order to enhance conductivity. Then as shown in block 1204, a spiked pulsed biphasic DC signal can be applied to the neck/head areas using a roller-type electrode, although it should be appreciated that other conductive electrodes can be used. The roller electrode should preferably be smooth enough, or rotate freely enough, to be maneuvered comfortably over the patient's neck area. As shown in block 1206, the electrode (probe) can be manipulated over the patient's neck for a predetermined time. Generally a 10-20 minute treatment is within a comfortable range for most patients. Finally, as shown in block 1208 the frequency and amplitude of the spiked signal can be adjusted during the course of treatment. Frequency can be varied within a 0.50-500 Hz range, and applied current during treatment can be up to about 600 microamps.

During the inventor's treatment of patients in a study, alternating microampere current at frequencies ranging from 0.5 to 100 Hz was directed through the fibrotic area using one stationary and one moveable electrode. The current source was an Electro-Myopulse 75F, a commercially available instrument, in mode 1 operated at the auto setting. Current was set as high as the patient could tolerate, typically at the maximum instrument setting of about 600 microamps.

During the first twenty minutes of each treatment session the fixed electrode was taped to the shoulder blade closest to the affected tissue. This electrode was a flat, square conducting plate of area 5×5cm². The movable electrode was a cylindrical roller, 7.6 cm in diameter and 7.6 cm long. The roller was repeatedly moved slowly from a region of healthy tissue just outside the fibrotic area into and across the region of scar tissue. For each patient all of the scar tissue related to radiation therapy was treated in this manner. Thus, if a supraclavicular radiation therapy field had been given in addition to the primary treatment fields, the supraclavicular area was included in the microcurrent treatment area.

During the next ten minutes the current source was the Electro-Acuscope 80L in mode 1 with settings of 10 Hz and 600 microamps. The single fixed electrode was replaced by two rectangular plates, each having an area of 10×27.2 cm², and connected to the current source through a preamplifier. The patient held one hand on each plate while the therapist treated the fibrotic area with the roller in the manner described above.

Twenty-six patients were treated twice per day, with a four to five hour interval between treatment sessions. A total of ten treatments were given over a period of five days. Subjective symptoms were recorded and range-of-motion measurements were made before the first treatment and at the end of each treatment day. Follow-up measurements and subjective assessments were made at one-month intervals for a total of three months. No additional microcurrent or physical therapy was permitted until the end of the three-month follow-up period.

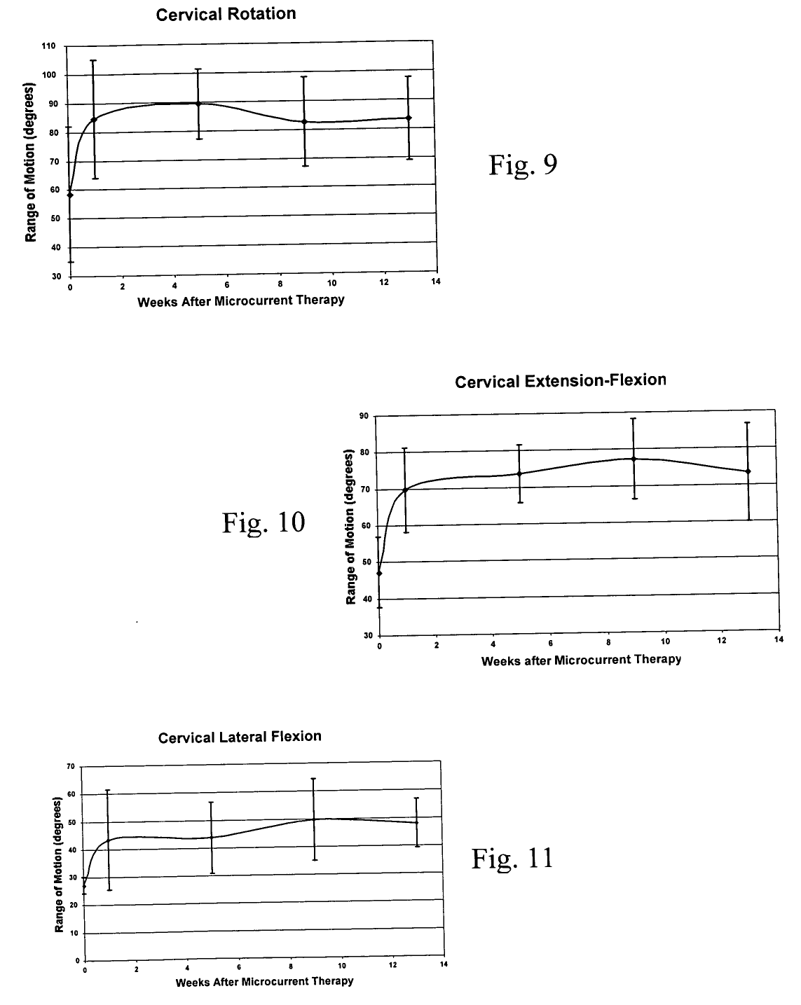

The range of right/left cervical rotation was compared to the nominal value of 170 degrees, which is considered normal for a healthy young individual. Ninety-two percent (24/26) of the patients exhibited improved cervical rotation at the end of microcurrent therapy. Of the twenty-two who returned for the three-month follow-up visit, three experienced continued improvement, while seventeen lost some of their range-of-motion, though their average mobility was somewhat better than it had been before microcurrent therapy. One patient in the mildly limited category experienced no improvement and one asymptomatic patient had measurements in the mildly limited category at the three-month follow-up. Referring to FIG. 9, a graph illustrates improvements for the three patients who started with severe limitations and completed all three follow-up visits on schedule.

Range of cervical extension/flexion was compared to the nominal value of 120 degrees, which is considered normal for a healthy young individual. Eighty-five percent (22/26) of the patients exhibited improved extension/flexion at the end of microcurrent therapy. Of the twenty-two who returned for the three-month follow-up visit, eight maintained or improved their end-of-treatment status. Ten of the twenty-two patients lost some range of motion but their mobility was still better than it had been before microcurrent therapy. The four patients who experienced no long-term improvement were already functioning within 80-90% of normal range. Referring to FIG. 10, a graph illustrates improvements for the three patients initially classified as most severely limited in extension/flexion.

Range of cervical right/left lateral flexion was compared to the nominal value of 90 degrees, which is considered normal for a healthy young individual. Eighty-one percent (21/26) of the patients exhibited improved range of lateral flexion at the end of microcurrent therapy. Of the twenty-two patients who returned for the three-month follow-up visit eight had continued to improve their range of motion without any additional therapy. Nine patients experienced a decrease compared to their ranges at the end of therapy but their mobility was still better than their measurements before therapy. Five patients experienced no long-term improvement. Referring to FIG. 11, a graph illustrates the improvements for the four patients who started with severe limitations and completed all three follow-up visits on schedule.

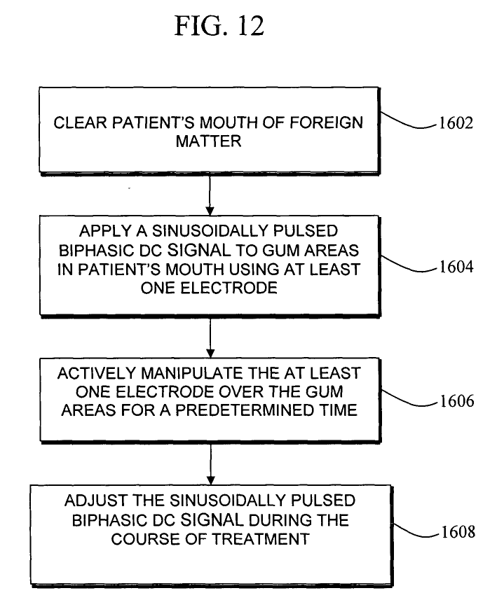

Referring to FIG. 12, a flow diagram sets forth steps that can be taken to apply intra-oral microcurrent therapy to patient suffering from xerostomia. Microcurrent therapy can be provided to a patient within the patient's mouth directly onto gums. A system such as that described in FIG. 5 can be used for delivering microcurrent up to about 200 microamps and at various frequencies (e.g., spiked/sinusoidal pulsed biphasic) in the range of 0.5 Hz to 500 Hz. Current above 200 microamps using a small probe can cause pain to patients because of the current density delivered over a small contact area. It should be appreciated to those skilled in the art that probes or conductive media providing larger contact areas can provide for higher currents, perhaps approaching 600 microamps.

As shown in block 1602 of FIG. 12, a patient's mouth must first be cleared of foreign matter (e.g., tobacco products, gum, removable orthodontics, or other medical devices. Then as shown in block 1604, a sinusoidally pulsed biphasic DC signal can be applied to the gum areas in the patient's mouth using at least one electrode. The electrode should preferably be small and smooth enough to be maneuvered comfortably over the gums of the patient. The areas targeted for treatment are just above the patient's upper teeth and just below the patient's lower teeth. As shown in block 1606, the electrode (probe) can be manipulated over the patient's gums for a predetermined time. Generally a 10-20 minute treatment is within a comfortable range for most patients. Finally, as shown in block 1608 the frequency and amplitude of the sinusoidal signal can be adjusted during the course of treatment. Frequency can be varied within a 0.5-500 Hz range, and applied current during treatment can be up to about 200 microamps in most cases.

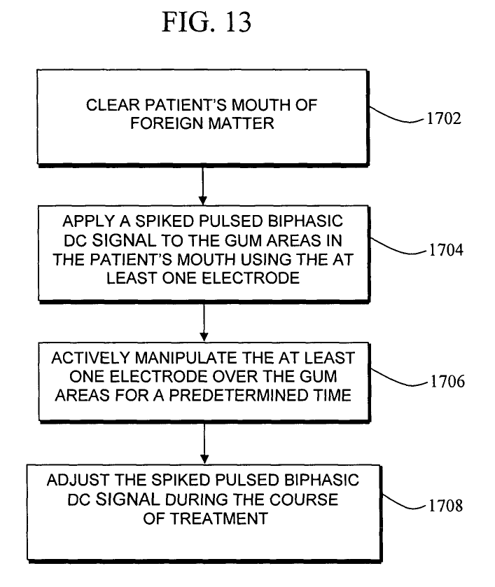

Referring to FIG. 13, another flow chart of a method for treating xerostomia is illustrated. As shown in block 1702, a patient's mouth must first be cleared of foreign matter (e.g., tobacco products, gum, removable orthodontics, or other medical devices. Then as shown in block 1704, a spiked pulsed biphasic DC signal can be applied to the gum areas in the patient's mouth using at least one electrode. The electrode should preferably be small and smooth enough to be maneuvered comfortably over the gums of the patient. The areas targeted for treatment are just above the patient's upper teeth and just below the patient's lower teeth. As shown in block 1706, the electrode (probe) can be manipulated over the patient's gums for a predetermined time. Again, generally a 10-20 minute treatment is within a comfortable range for most patients. Finally, as shown in block 1708 the frequency and amplitude of the spiked signal can be adjusted during the course of treatment. Frequency can be varied within a 10-500 Hz range, and applied current during treatment can be up to about 200 microamps in most cases.

In head-and-neck cancer patients, radiation-induced fibrosis can lead to many different complaints, depending on the size and placement of treatment fields, the total dose, and whether the patient also had surgery. Limitations in neck range-of-motion are common and are quantifiable. Because this study was looking for objectively measured changes associated with microcurrent therapy, the protocol was designed to achieve improvement in range of motion. Measurements were made on all patients in the study regardless of whether the patient considered range-of-motion limitations to be a problem. In fact, most of the patients in the mildly and moderately limited groups had learned to compensate for the limitations and were surprised when measurements showed how much capability they had lost. As could be expected, the patients who were most severely limited received the greatest degree of benefit.

Patients also received relief from a number of complaints that were not directly targeted in the treatment protocol, the most significant of which were trismus (limited mouth opening) and xerostomia.

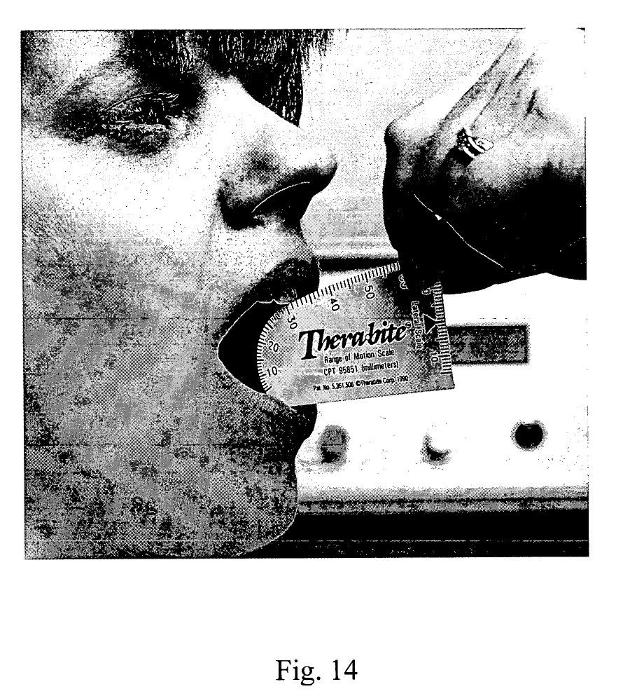

Oral opening was measured using a Therabite™ scale (manufactured and supplied by Therabite Corp.) as shown in FIG. 14. The measurement was made for all 26 patients, even if trismus was not a complaint. Eighty-one percent (21/26) of the patients exhibited improved oral opening after impedance-controlled microcurrent therapy. It should be noted that only 16 of the 26 patients stated that trismus was a problem. Four of the sixteen showed no improvement during the course of the study. One had no improvement at the end of the treatment week but had gained 3 mm in oral opening at the end of three months. For the seven patients who maintained improvement in oral opening the average increase was 4.6±2.2 mm three months after the end of microcurrent therapy.

Sixteen patients with xerostomia were treated using the Electro-Myopulse 75F and Electro-Acuscope 80L, which are commercially available instruments. All patients had received a full course of either photon or neutron radiation as treatment of a malignancy of the head and neck. All were at least six months post radiation therapy and had no evidence of disease. External electrodes were used to administer microcurrent therapy twice per day for five consecutive days. Saliva production was quantified by weighing the saliva each patient was able to expectorate into a paper cup during a five-minute period. Both un-stimulated saliva production (USP) and stimulated saliva production (SSP) rates were obtained, with concentrated lemon juice used as a stimulating agent. Data were collected before the first microcurrent treatment, after ten treatments, and monthly during the three-month follow-up period.

At the conclusion of five treatment days, 81% of the patients (13/16) experienced an increase in USP. Twelve of these patients also experienced an increase in SSP. The increases in mean USP and SSP rates were 56% and 42%, respectively. During the three-month follow-up period patients received no additional microcurrent therapy. Of the fifteen who returned for follow-up after three months, 11/15 and 12/15 had higher USP and SSP rates, respectively than their pre-microcurrent baseline rates. The improvement for the mean USP was 104%, while the mean SSP was 38% greater than baseline. For some of these patients, (10/15) and (7/15), the USP and SSP rates were higher than their end-of-treatment rates, indicating continued improvement during the follow-up period. No patients experienced any untoward effects.

TABLE 1
List of subjective complaints. Denominator indicates number of
patients reporting a symptom. Numerator is the number who reported
an improvement in the symptom after impedance-controlled
microcurrent therapy.

| | Percentage reporting |
|---|---|
| Symptom | improvement. |
| Tongue immobility | 3/8 = 37% |
| Impaired speech | 3/6 = 50% |
| Stiffness discomfort | 24/26 = 92% |
| Facial asymmetry | 6/7 = 86% |
| Soft tissue edema | 11/17 = 65% |
| Trismus | 10/16 = 62% |
| Dry mouth | 15/20 = 75% |
| Difficulty swallowing | 4/10 = 40% |
| Cervical/facial spasms | 10/12 = 83% |
| Fibrosis | 12/20 = 60% |
| Inability to purse lips | 5/5 = 100% |
| Difficulty breathing | 3/3 = 100% |
| Tenderness | 10/15 = 67% |
| Pain | 9/13 = 69% |
| Numbness | 6/8 = 75% |

Many of the benefits observed at the end of the treatment week were sustained. In some cases there was continued improvement during the three-month follow-up period suggesting that the treatment had initiated tissue repair. These observations support the view that microcurrent therapy can initiate long-term benefit for patients suffering from fibrosis.

1. Method of applying biphasic DC signal at various frequencies, durations and/or amplitudes to areas of a cancer patient's body being targeted for microcurrent therapy, comprising the steps of:
apply a biphasic DC signal to an area of a cancer patient's body targeted for microcurrent treatment using at least one electrode;
actively manipulate said at least one electrode to said area of a cancer patient's body for a predetermined time; and
adjust the biphasic DC signal during said predetermined time.
wherein said method is provided to a patient at least one of prior to the patient undergoing cancer treatment or concurrently with a patient undergoing cancer treatment.

2. The invention of claim 1 wherein said method is also provided after a patient receives cancer treatment.

3. The method of claim 1 wherein microcurrent is provided to a patient at frequencies in the range of about 0.5 Hz to about 500 Hz.

4. The method of claim 1 wherein microcurrent is provided to a patient in the range of about 25 microamps to about 600 microamps.

5. The method of claim 1 wherein microcurrent is provided to a patient for a period up to about 60 minutes.

6. The invention of claim 1, further comprising the steps of:
receiving impedance matching information through said microcurrent therapy system during said predetermined time;
analyzing said impedance matching information to identify impedance variations between said system and said patient's body; and
adjusting the biphasic DC signal to compensate for impedance variations between said microcurrent therapy system and the patient.

7. The invention of claim 6 wherein said method is provided prior to a patient undergoing cancer treatment.

8. The invention of claim 6 wherein said method is provided concurrent with a patient undergoing cancer treatment.

9. The invention of claim 6 wherein said method is provided after a patient receives cancer treatment.

10. Methods of applying biphasic DC signal at various frequencies, durations and/or amplitudes to areas of a cancer patient's body being targeted for microcurrent therapy, comprising the steps of:
applying a biphasic DC signal from a microcurrent therapy system to an area of a cancer patient's body targeted for microcurrent treatment using at least one electrode;
actively manipulating said at least one electrode to said area of a cancer patient's body for a predetermined time;
receiving impedance matching information through said microcurrent therapy system during said predetermined time;
analyzing said impedance matching information to identify impedance variations between said system and said patient's body; and
adjusting the biphasic DC signal in response to said impedance variations.

11. The method of claim 10 wherein microcurrent is provided to a patient at frequencies in the range of about 0.5 Hz to about 500 Hz.

12. The method of claim 10 wherein microcurrent is provided to a patient in the range of about 25 microamps to about 600 microamps.

13. The method of claim 10 wherein microcurrent is provided to a patient for a period up to about 60 minutes.

14. The invention of claim 10 wherein said method is provided prior to a patient undergoing cancer treatment.

15. The invention of claim 10 wherein said method is provided concurrent with a patient undergoing cancer treatment.

16. The invention of claim 10 wherein said method is provided after a patient receives cancer treatment.

17. A system for applying biphasic DC signal at various frequencies, durations and/or amplitudes to areas of a cancer patient's body being targeted for microcurrent therapy, comprising:
a pulsed biphasic DC generator in electrical communication with a controller and at least one electrode;
a user interface, said user interface for enabling a user to provide manual settings and adjustments of biphasic DC signals provided by said system;
a controller, said controller adapted to receives manual setting from said user interface and further adapted to automatically adjust biphasic DC signal attributes including amplitude and frequency in response to measurements obtained from a feedback module; and
a feedback module in communication with said controller, said feedback module adapted to measure impedance variations between said system and a cancer patient during microcurrent therapy, wherein said impedance variations are obtainable by said controller.

18. The system of claim 17, said at least one electrode includes at least one of a: probe, roller, plate, cuff, clamp, sheet, and liquid.

19. The system of claim 17 wherein said at least one electrode is adapted to apply microcurrent to a patient at at least one of: large bodily areas, small bodily areas, crevicular bodily areas, and intra-oral bodily areas.

20. The system of claim 19, said at least one electrode comprising of at least one of a: probe, roller, plate, cuff, clamp, sheet, and liquid.

21. The invention of claim 10 wherein said system is used for the application of biphasic DC signals at various frequencies, durations and/or amplitudes prior to a patient undergoing cancer treatment.

22. The invention of claim 10 wherein said system is used for the application of biphasic DC signals at various frequencies, durations and/or amplitudes concurrently with a patient undergoing cancer treatment.

23. The invention of claim 10 wherein said system is used for the application of biphasic DC signals at various frequencies, durations and/or amplitudes after a patient receives cancer treatment.
